(19) United States
(12) Patent Application Publication
(10) Pub. No.: US 2005/0228450 A1
(43) Pub. Date: Oct. 13, 2005

(54) Measuring and stimulating device using electronic signal

(76) Inventor: Seung-Kee Mo, Seoul (KR)

Correspondence Address: PERKINS COIE LLP;PATENT-SEA, P.O. BOX 1247, SEATTLE, WA, 98111-1247, US

(21) Appl. No.: 10/521,873

(22) PCT Filed: Jul. 3, 2003
(86) PCT No.: PCT/KR03/01311

§ 371 Date: Jan. 20, 2005

(30) Foreign Application Priority Data
Jul. 22, 2002 (KR) 1020020042874

(51) Int. Cl. A61N 1/00

(52) U.S. Cl. 607/2

(57) ABSTRACT

This invention relates to measuring and stimulating device using electrical signals. The measuring and stimulating device comprises at least two conductive members made of material having electrical conductivity, a connection member for transferring the relevant electrical signal to each of the at least two conductive members, and a control unit for transmitting a test electrical signal to the at least two conductive members, receiving measured electrical signals corresponding to the test electrical signal from the at least two conductive members, and transmitting a body electrical signal to the at least two conductive members.

### BACKGROUND OF THE INVENTION

1. Field of the Invention

The present invention relates to a measuring and stimulating device using electrical signals, and specifically, to a measuring and stimulating device using electrical signals, which measures electromyogram of a human body using electrical signals and provides electrical stimulation to the human body with the electrical current corresponding to the measured electromyogram.

2. Description of the Related Art

Most of disease therapies may be classified as drugs, operations or the like. However, the treatments with drugs (medication) may cause unexpected side effects due to long-term treatment, and the treatments with operations may raise patient's detestation or trauma for the treatment type and require high cost.

In recent, methods of treating a human body using electrical signals have been widely used, the methods being non-invasive and having less side effects, unlike the conventional treatments.

Electrical stimulation is a method of delivering electrical signals through electrode patches attached on the surface of a skin. As a result, muscles repeat contraction and relaxation which are effects of the physical exercise. Therefore, the exercise accelerates recovery of pain.

In addition, such methods using electrical signals are used for reducing fat as well as treating a human body.

Electrode patches to which electrically conductive gel is applied is used as the representative conductive member in the conventional method of treating a human body using electrical signals. However, there are problems that a plurality of electrode patches should be attached and these electrode patches are disposable.

Further, the conventional device for treating a human body using electrical signals has a problem that the human body may be in danger of a burn due to the concentration of currents because the conductive member is not in a good contact with a skin.

Furthermore, the conventional device for treating a human body using electrical signals cannot supply suitable intensity of electrical stimulation to the human body, though the suitable intensity of electrical stimulation is different every human body.

### SUMMARY OF THE INVENTION

Therefore, it is an object of the present invention to provide a measuring and stimulating device using electrical signals which measures electromyogram of a human body and supplies electrical stimulation to the human body.

It is another object of the present invention to provide a measuring and stimulating device using electrical signals, capable of being in good contact with a human body to supply electrical signals to the human body.

It is still another object of the present invention to provide a measuring and stimulating device using electrical signals, comprising a conductive member being semi-permanent, not disposable.

It is still another object of the present invention to provide a measuring and stimulating device using electrical signals, capable of supplying electrical stimulation the most suitable for a human body.

In order to accomplish the above objects, according to one preferred embodiment of the present invention, a stimulating device using electrical signals, which is attached to a human body for use, is provided, the device comprising: a main body made of non-conductive material; at least two conductive members attached to the surface of the main body and made of conductive material; and a connecting section for transferring the respective electrical signals to the at least two conductive members.

The main body may be a band type, and a control unit for controlling supply of the electrical signals to the connecting section may be further comprised.

It is preferable that the connecting section and the control unit are coupled through at least one of a wireless connection and a wire connection, and the non-conductive material and the conductive material are alternately arranged.

It is preferable that the non-conductive material and the conductive material are separately arranged in a longitudinal or lateral direction, and examples of the conductive material include silicon, rag, cloth and leather having electrical conductivity.

Examples of the non-conductive material may include silicon, rag, cloth and leather having electrical non-conductivity.

It is preferable that the conductive material having the plus (+) polarity and the conductive material having the minus (−) polarity are arranged alternately.

It is preferable that one end of the conductive member is electrified, the other end of the conductive member is insulated, the connecting section and the conductive member are connected each other through a connection member, and one end of the connection member comprises at least one of a lead terminal and an extruded terminal.

It is preferable that a plurality of grooves may be formed separately in the main body, the conductive members may be coupled to the main body correspondingly to the grooves, and height of the conductive member is equal to or greater than depth of the groove.

It is preferable that the main body further comprises a Velcro for adjusting a size of circumference, when the main body is a band type.

The control unit may comprise a signal processing section for identifying signals and an adjusting section for controlling the signals.

According to another preferred embodiment of the present invention, a measuring and stimulating device using electrical signals, which is attached to a human body for use, is provided, the device comprising: at least two conductive members made of conductive material; a connecting section for transferring the respective electrical signals to the at least two conductive members; and a control unit coupled to the connecting section, for supplying a test electrical signal to the conductive members, receiving a measured electrical signal corresponding to the test electrical signal, and supplying a body electrical signal corresponding to the measured electrical signal.

It is preferable that the main body is a band type, and the connecting section and the control unit may be coupled through at least one of a wireless connection and a wire connection.

Furthermore, the device may further comprise a main body made of non-conductive material, wherein the conductive members are attached to one surface of the main body, and it is preferable that the non-conductive material and the conductive material are separately arranged in a longitudinal or lateral direction.

Examples of the non-conductive material may include silicon, rag, cloth and leather having electrical non-conductivity, and it is preferable that a plurality of grooves are formed separately in the main body and the conductive members are coupled to the main body correspondingly to the grooves.

It is preferable that height of the conductive member is equal to or greater than depth of the groove, and the conductive material may include at least any one of silicon, rag, cloth and leather having electrical conductivity.

It is preferable that the conductive material having the plus (+) polarity and the conductive material having the minus (−) polarity are arranged alternately, and it is also preferable that one end of the conductive member is electrified and the other end of the conductive member is insulated.

It is preferable that the connecting section and the conductive member are connected each other through a connection member, and one end of the connection member comprises at least one of a lead terminal or an extruded terminal. The main body may further comprise a Velcro for adjusting a size of circumference, when the main body is a band type.

### BRIEF DESCRIPTION OF THE DRAWINGS

FIG. 1 is a block diagram illustrating a measuring and stimulating device using electrical signals according to one preferred embodiment of the present invention;

FIG. 2 is a block diagram of a control unit according to one preferred embodiment of the present invention;

FIG. 3 is a constructional view of a measuring and stimulating unit according to one preferred embodiment of the present invention;

FIG. 4 is a vertical cross-sectional view illustrating a state in which a primary main body and a conductive member are coupled each other according to one preferred embodiment of the present invention;

FIGS. 5A and 5B are views a method of connecting the conductive member and a connection member each other according to one preferred embodiment of the present invention;

FIGS. 6A, 6B and 6C are views illustrating states in which the measuring and stimulating unit of the measuring and stimulating device using electrical signals according to one preferred embodiment of the present invention is coupled to a human body, respectively; and

FIG. 7 is a horizontal cross-sectional view of a measuring and stimulating unit when the main body is a band type according to one preferred embodiment of the present invention.

### DESCRIPTION OF THE PREFERRED EMBODIMENTS

Now, preferred embodiments of the present invention will be described in detail with reference to the appended drawings.

FIG. 1 is a block diagram illustrating a measuring and stimulating device using electrical signals according to one preferred embodiment of the present invention.

Referring to FIG. 1, a control unit 1 and a measuring and stimulating unit 3 of the measuring and stimulating device according to the preferred embodiment of the present invention are coupled to each other through a wireless or wire 5.

The control unit 1 serves for controlling transmitting/receiving types of electrical signals with the measuring and stimulating unit 3. Specifically, the control unit 1 serves for supplying a test electrical signal to the measuring and stimulating unit 3, receiving a measured electrical signal corresponding to the test electrical signal, and supplying a body electrical signal corresponding to the measured electrical signal.

Here, the test electrical signal is a signal for measuring electromyogram of a human body, and the measured electrical signal is a signal indicative of the electromygram of a human body measured in accordance with the test electrical signal. Further, the body electrical signal is a signal for applying the most suitable electrical stimulation to the human body.

The measuring and stimulating unit 3 serves for supplying the test electrical signal supplied from the control unit 1 to the human body, receiving the electrical signal measured from the human body, and supplying the measured electrical signal to the control unit 1. Further, the measuring and stimulating unit 3 receives the body electrical signal from the control unit 1 and supplies the body electrical signal to the human body.

The measuring and stimulating unit 3 can be formed in a band type in order to be in a good contact with the human body when the body electrical signal is supplied to the human body.

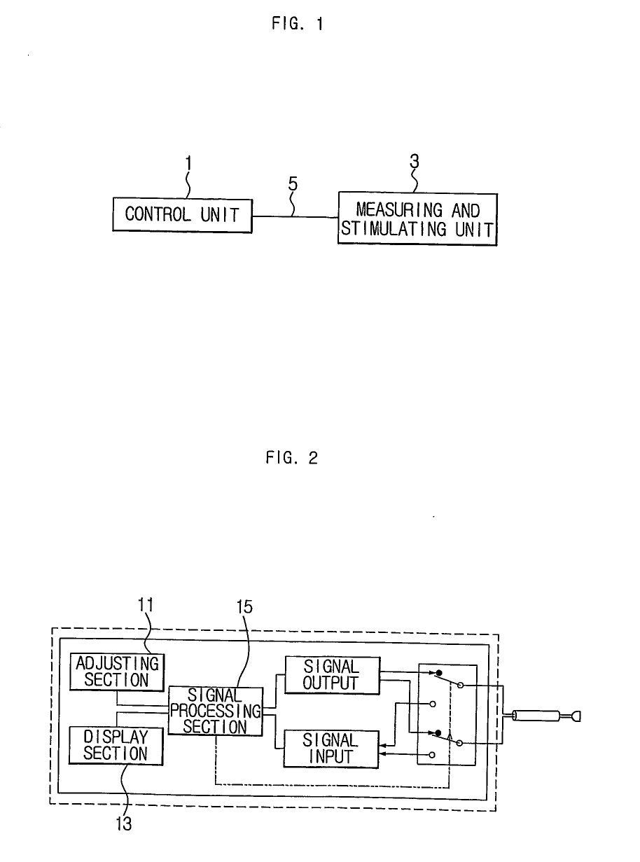

FIG. 2 is a block diagram of the control unit according to one preferred embodiment of the present invention.

Referring to FIG. 2, the control unit 1 comprises an adjusting section 11, a display section 13 and a signal processing section 15.

The adjusting section 11 serves for controlling the electrical signals to be supplied to the measuring and stimulating unit 3. The adjusting section 11 may be at least one of a switch or a dial.

The display section 13 serves for displaying an amount of electrical signal supplied to the measuring and stimulating unit 3 or received from the measuring and stimulating unit 3, or displaying a kind of electrical signal identified by the signal processing section 15. According to one preferred embodiment of the present invention, the display section 13 may comprise at least one of LED and LCD for displaying the identification results of the kind of electrical signal.

The signal processing section 15 serves for processing the electrical signals through adjustment of the adjusting section 11, and controlling the output of signals or identifying the signals to be input.

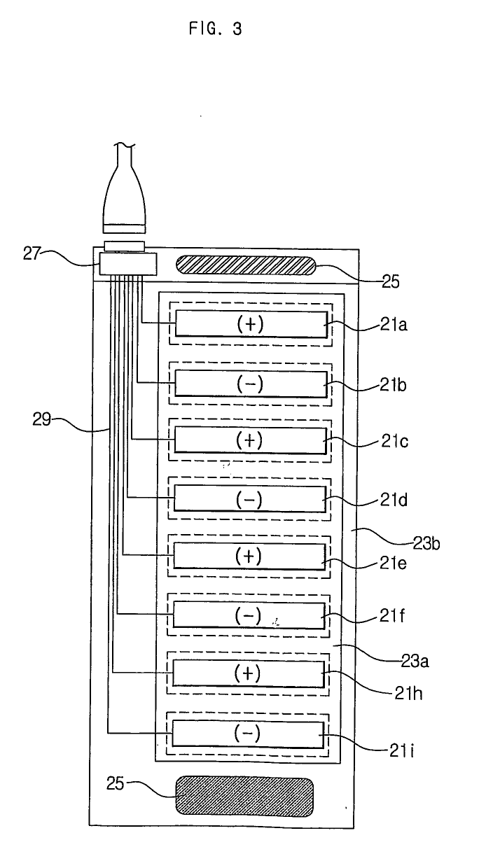

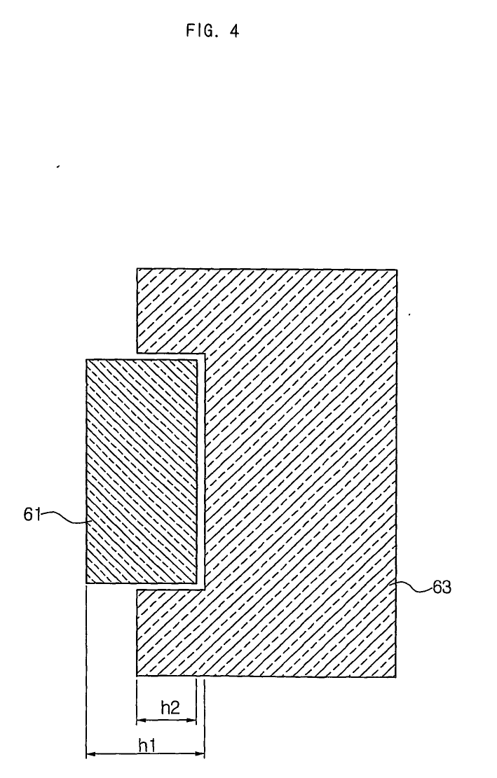

FIG. 3 is a constructional view of the measuring and stimulating unit according to one preferred embodiment of the present invention, and FIG. 4 is a vertical cross-sectional view illustrating a state in which a primary main body and a conductive member are coupled each other according to one preferred embodiment of the present invention.

Referring to FIG. 3, the measuring and stimulating unit 3 comprises conductive members 21a to 21j (hereinafter, generally referred to as 21), main bodies 23a, 23b, a Velcro 25, a connecting section 27 and connecting members 29.

The conductive members 21 serve for supplying the test electrical signals and the body electrical signals to a human body, or receiving the measured electrical signals from the human body.

The conductive members 21 are made of a conductive material. The conductive material may be any one of silicon, rag, cloth and leather. Further, the conductive members have a plus (+) polarity 21a, 21c, 21e, 21h and a minus (−) polarity 21b, 21d, 21f, 21j. Furthermore, the conductive members having different polarities are arranged alternately.

The main bodies 23a, 23b comprises a primary main body 23a and a secondary main body 23b. The respective main bodies 23a, 23b serve for supporting the conductive members. Specifically, the conductive members 21a, 21b are coupled to one surface of the primary main body 23a spaced from each other, and the secondary main body 23b supports the primary main body 23a and the conductive members 21a, 21b. As a result, the conductive members 21a, 21b can be in a good contact with a human body to supply the electrical signals to the human body. A coupling example of the primary main body 23a and the conductive members 21 will be described specifically with reference to FIG. 4.

Referring to FIG. 4, a plurality of grooves are separately formed in the primary main body 63 to facilitate the coupling to the conductive members 61. The conductive members 61 are coupled to the grooves of the primary main body 63, and height h1 of the conductive members 61 is equal to or greater than depth h2 of the grooves of the primary main body 63. Accordingly, the conductive members 61 of the measuring and stimulating unit can be in a good contact with the human body to supply the electrical signals to the human body.

As described above, the conductive members 21 of the measuring and stimulating unit according to the present invention is constructed to be in a good contact with the human body and supply the electrical signals to the human body in this state.

When the conductive members 21 are not in a good contact with the human body and thus an area of the conductive members in contact with a human body is small, the human body can be subjected to a burn due to concentration of electrical signals.

In order to solve this problem, according to one preferred embodiment of the present invention, the main bodies 23a, 23b may be formed in a band type having a good elasticity. Since the band type main bodies 23a, 23b form pressure for bringing the conductive members 21 into a good contact with a skin, the area of the conductive members 21 in contact with the human body is increased. As a result, a contact resistance of the conductive members 21 and the human body is decreased, thereby allowing the electrical signals to flow easily.

Furthermore, the conductive members 21 may be made of conductive silicon having a low hardness and thus having a good flexibility.

The main bodies 23a, 23b is made of non-conductive material. The non-conductive material may be at least one of silicon, rag, cloth and leather having non-conductivity.

The Velcro 25 serves for adjusting a size of a circumference when the measuring and stimulating unit is a band type. Therefore, using the Velcro 25, the measuring and stimulating unit 3 can get in a good contact with portions of a human body having different sizes.

The connecting section 27 is coupled to the control unit through at least one of a wireless connection and a wire connection, to serve for transferring the electrical signals to at least two conductive members (for example, 21a, 21b), respectively.

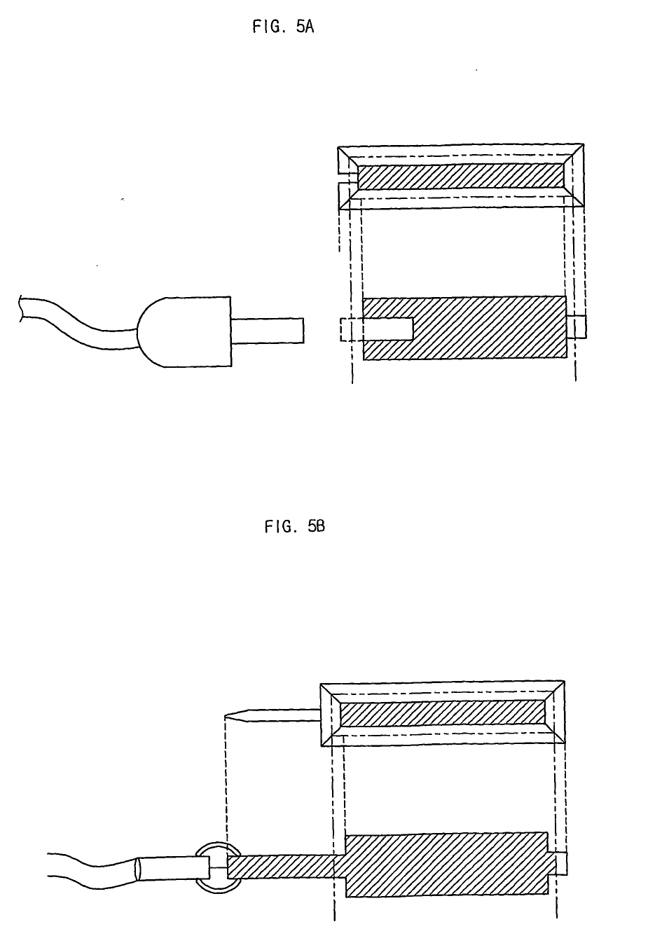

In addition, the connecting section 27 is connected to the conductive members 21 through the connection members 29. A method of connecting a connection member 29 and a conductive member 21 each other will be described specifically with reference to FIGS. 5A and 5B.

FIGS. 5A and 5B are views the method of connecting the conductive member and the connection member each other according to one preferred embodiment of the present invention.

Referring to FIGS. 5A and 5B, one end of the connection member may be connected to a lead terminal as shown in FIG. 5A or an extruded terminal shown in FIG. 5B.

Here, one end of the conductive member connected to the connection member is electrified, and the other end thereof is insulated.

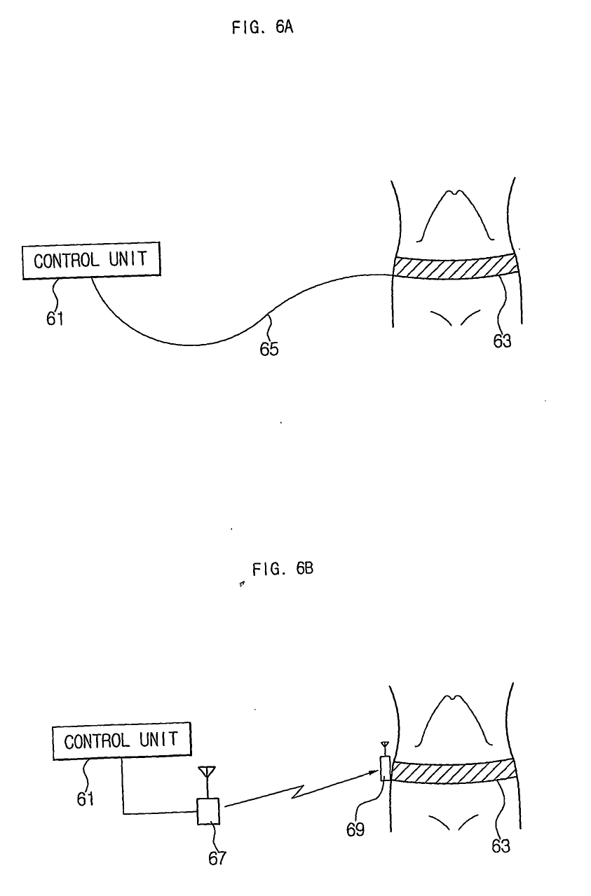

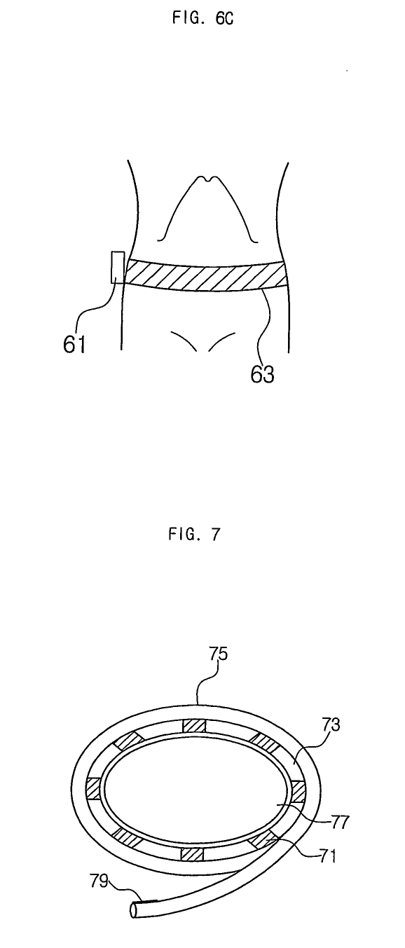

FIGS. 6A, 6B and 6C are views illustrating states in which the measuring and stimulating unit of the measuring and stimulating device using electrical signals according to one preferred embodiment of the present invention is coupled to a human body, respectively.

FIGS. 6A, 6B and 6C show a case in which the main body of the measuring and stimulating device using electrical signals is a band type.

FIG. 6A is a view illustrating a case in which the control unit 61 and the measuring and stimulating unit 63 are coupled with a wire connection 65. Here, the wire connection is not limited, only if it can supply electrical signals to a human body and be reduced to practice by those skilled in the art. A specific example of the wire connection includes a cable, an electric wire or the like.

FIG. 6B is a view illustrating a case in which the control unit 61 and the measuring and stimulating unit 63 are coupled to each other with wireless transmitting/receiving units 67, 69.

Since the wireless transmitting/receiving units 67, 69 can be obviously considered by those skilled in the art, description of the wireless transmitting/receiving units will be omitted.

FIG. 6C is a view illustrating a case in which the control unit 61 and the measuring and stimulating unit 63 are coupled into one body. Like above, if the control unit 61 and the measuring and stimulating unit 63 are coupled directly, the cable or the electric wire is not necessary and it is easy to carry it.

FIG. 7 is a horizontal cross-sectional view of the measuring and stimulating unit when the main body is a band type according to one preferred embodiment of the present invention.

Referring to FIG. 7, the conductive members 71 made of conductive material and the primary main bodies 73 made of non-conductive material are separately arranged, respectively, and the secondary main body 75 supports the conductive members 71 and the primary main bodies 73.

On the other hand, the main bodies 73, 75 are a band type, thereby applying pressure to be in a good contact with a human body. In addition, the Velcro 79 can be adjusted such that the size of circumference is varied with portions of the human body 77, the measuring and stimulating unit can be in a good contact with the human body.

Although the present invention has been described in connection with the preferred embodiments, it will be understood by those skilled in the art that various changes or modifications may be made thereto without departing from the spirit and scope of the present invention.

### INDUSTRIAL AVAILABILITY

According to the present invention, it is possible to provide the measuring and stimulating device using electrical signals for measuring electromyogram of a human body using the electrical signals and supplying the electrical stimulation the most suitable for the human body.

It is also possible to provide the measuring and stimulating device using electrical signals capable of being in good contact with a human body to supply electrical signals to the human body, thereby preventing a burn of the human body due to the concentration of current.

It is also possible to provide the measuring and stimulating device using electrical signals, capable of reducing cost by using the conductive members being semi-permanent, not disposable.

It is also possible to provide the measuring and stimulating device using electrical signals, capable of supplying electrical stimulation most suitable for a human body, thereby not applying large damage to the human body.

1. A stimulating device using electrical signals comprising:
a main body made of non-conductive material;
at least two conductive members attached to a surface of the main body and made of conductive material; and
a connecting section for transferring the respective electrical signals to the at least two conductive members.

2. The stimulating device using electrical signals according to claim 1, wherein said main body is a band type.

3. The stimulating device using electrical signals according to claim 1, further comprising a control unit for controlling supply of the electrical signals to the connecting section.

4. The stimulating device using electrical signals according to claim 3, wherein said connecting section and said control unit are coupled through at least one of a wireless connection and a wire connection.

5. The stimulating device using electrical signals according to claim 3, wherein said control unit comprises:
a signal processing section for identifying signals; and
an adjusting section for controlling the signals.

6. The stimulating device using electrical signals according to claim 1, wherein the non-conductive material and the conductive material are separately arranged in a longitudinal or lateral direction.

7. The stimulating device using electrical signals according to claim 1, wherein said conductive material includes at least any one of silicon, rag, cloth and leather having electrical conductivity.

8. The stimulating device using electrical signals according to claim 1, wherein said non-conductive material includes at least any one of silicon, rag, cloth and leather having electrical non-conductivity.

9. The stimulating device using electrical signals according to claim 1, wherein said conductive material of the conductive members comprises a plus (+) polarity or a minus (−) polarity, and the conductive material having the plus (+) polarity and the conductive material having the minus (−) polarity are arranged alternately.

10. The stimulating device using electrical signals according to claim 1, wherein one end of the conductive member is electrified, and the other end of the conductive member is insulated.

11. The stimulating device using electrical signals according to claim 1, wherein said connecting section said the conductive member are connected each other through a connection member, and one end of the connection member comprises at least one of a lead terminal or an extruded terminal.

12. The stimulating device using electrical signals according to claim 1, wherein a plurality of grooves are formed separately in the main body, and the conductive members are coupled to the main body correspondingly to the grooves.

13. The stimulating device using electrical signals according to claim 12, wherein height of the conductive member is equal to or greater than depth of the groove.

14. The stimulating device using electrical signals according to claim 1, further comprising a Velcro for adjusting a size of circumference, when the main body is a band type.

15. The stimulating device using electrical signals according to claim 1, wherein said non-conductive material and said conductive material are alternately arranged.

16. A measuring and stimulating device using electrical signals comprising:
at least two conductive members made of conductive material;
a connecting section for transferring the respective electrical signals to the at least two conductive members; and
a control unit coupled to the connecting section, for supplying a test electrical signal to the conductive members, receiving a measured electrical signal corresponding to the test electrical signal, and supplying a body electrical signal corresponding to the measured electrical signal.

17. The measuring and stimulating device using electrical signals according to claim 16, further comprising a main body made of non-conductive material,
wherein the conductive members are attached to one surface of the main body.

18. The measuring and stimulating device using electrical signals according to claim 16, wherein said connecting section and said control unit are coupled through at least one of a wireless connection and a wire connection.

19. The measuring and stimulating device using electrical signals according to claim 17, wherein said main body is a band type.

20. The measuring and stimulating device using electrical signals according to claim 17, wherein said non-conductive material and said conductive material are separately arranged in a longitudinal or lateral direction.

21. The measuring and stimulating device using electrical signals according to claim 17, wherein said non-conductive material includes at least any one of silicon, rag, cloth and leather having electrical non-conductivity.

22. The measuring and stimulating device using electrical signals according to claim 17, further comprising a Velcro for adjusting a size of circumference, when the main body is a band type.

23. The measuring and stimulating device using electrical signals according to claim 17, wherein a plurality of grooves are formed separately in the main body, and the conductive members are coupled to the main body correspondingly to the grooves.

24. The measuring and stimulating device using electrical signals according to claim 23, wherein height of the conductive member is equal to or greater than depth of the groove.

25. The measuring and stimulating device using electrical signals according to claim 16, wherein said conductive material includes at least any one of silicon, rag, cloth and leather having electrical conductivity.

26. The measuring and stimulating device using electrical signals according to claim 16, wherein said conductive material of the conductive members comprises a plus (+) polarity or a minus (−) polarity, and the conductive material having the plus (+) polarity and the conductive material having the minus (−) polarity are arranged alternately.

27. The measuring and stimulating device using electrical signals according to claim 16, wherein one end of the conductive member is electrified, and the other end of the conductive member is insulated.

28. The measuring and stimulating device using electrical signals according to claim 16, wherein said connecting section and said conductive member are connected each other through a connection member, and one end of the connection member comprises at least one of a lead terminal or an extruded terminal.
